(19) United States
(12) Patent Application Publication
(10) Pub. No.: US 2002/0032220 A1
(43) Pub. Date: Mar. 14, 2002

(54) Formulations comprising dissolved paroxetine

(75) Inventors: Ahmad Khalaf Al-Deeb Al-Ghazawi, Waltham (GB); Graham Stanley Leonard, St Albans (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham plc

(21) Appl. No.: 09/925,354

(22) Filed: Aug. 9, 2001

Related U.S. Application Data
(63) Continuation of application No. 09/554,861, filed on Jun. 29, 2000, now abandoned.

(30) Foreign Application Priority Data
Nov. 21, 1997 (GB) 9724544.3

(51) Int. Cl. A61K 9/48; A61K 31/4525

(52) U.S. Cl. 514/321; 424/451

(57) ABSTRACT

Pharmaceutical formulations of paroxetine are provided in which the paroxetine is in solution in a solid, semi-solid or liquid carrier. The solutions are used to fill capsules, or self-supporting solid solutions are shaped into solid dosage forms such as tablets or pellets. Also disclosed are novel liquid formulations in which a solubilising agent is used to solubilise paroxetine in oils and/or lipids, and methods of avoiding other paroxetine forms converting to the hemihydrate, by use of anhydrous or hydrophobic carriers or excipients.

The present invention relates to novel formulations of a pharmaceutically active compound, and to the use of the formulations in therapy. In particular this invention is concerned with new formulations of the anti-depressant paroxetine.

Pharmaceutical products with antidepressant and anti-Parkinson properties are described in U.S. Pat. No. 3,912,743 and U.S. Pat. No. 4,007,196. An especially important compound among those disclosed is paroxetine, the (−)trans isomer of 4-(4′-fluorophenyl)-3-(3′,4′-methylenedioxy-phenoxymethyl)-piperidine. Paroxetine hydrochloride hemihydrate is used in therapy for the treatment and prophylaxis of inter alia depression, obsessive compulsive disorder (OCD) and panic.

Paroxetine hydrochloride hemihydrate is described in EP-A-0223403 of Beecham Group and paroxetine hydrochloride anhydrate Forms A, B, C and D are described in WO 96/24595 of SmithKline Beecham plc. All solid oral dosage forms of paroxetine hydrochloride sold to date have been in the form of oral swallow tablets, containing the hemihydrate. WO 95/16448 discloses that paroxetine is likely to develop a pink colour unless it is formulated into tablets using a formulation process in which water is absent, such as dry direct compression of paroxetine or dry granulation of paroxetine followed by compression into tablets.

To assist in patient compliance with dosace regimes, there remains a need for alternative dosage forms to the swallow tablet. However, the low solubility of paroxetine hydrochloride in many solvents has made this difficult to achieve. In particular, it was not believed feasible to devise an oral swallow capsule of sufficiently small size to be readily swallowed and containing sufficient paroxetine in solution for an effective dose, using physiologically acceptable solvents capable of encapsulation. The present inventors have now overcome this problem.

In one aspect, the present invention provides an oral swallow capsule containing paroxetine dissolved in a carrier.

Typically the oral swallow capsule comprises a capsule shell containing paroxetine as the free base or a pharmaceutically acceptable salt or solvate thereof in solution in a carrier. The carrier may be liquid or solid.

A liquid carrier may be a solvent present in the capsule as a flowable liquid, as a viscous liquid or semi-solid or as a gel. The carrier may also be a solid or semi-solid solvent such as fats and waxes, or film-forming or thermoplastic polymers. Solvents in which supersaturated solutions can be formed are advantageous because of the possibility to increase the loading of active ingredient.

When the carrier is a solid or semi-solid or a gel, the paroxetine containing carrier may be self-supporting without encapsulation. Accordingly a self-supporting formulation may be encapsulated by other means than loading into a preformed capsule shell, for example by coating with an encapsulating material. Also the self-supporting formulation may be used as a dosage form without encapsulation.

Accordingly in another aspect the present invention provides an oral swallow solid dosage form containing paroxetine dissolved in a solid, semi-solid or gel carrier.

Typically the solid dosage form comprises tablets, pellets, spheroids, granules, lozenges or gels in which paroxetine is present as a solid solution in a polymeric carrier.

Capsules and solid dosage forms of this invention may be coated to assist in administration of the active ingredient, for example using an enteric coating material to prevent release of paroxetine in the stomach, coatings to delay or control release of paroxetine and coatings of taste-masking agents. Alternatively such materials can be incorporated in the carrier to achieve the same effect.

The paroxetine is preferably used as the hydrochloride, and as such may be used as the hemihydrate, or as the anhydrate Form A, B, C or D, or as any other form of paroxetine hydrochloride or paroxetine, such as pharmaceutically acceptable salts other than the hydrochloride. Other suitable paroxetine forms include paroxetine free base, and amorphous and non-crystalline forms of paroxetine and pharmaceutically acceptable derivatives of paroxetine.

In a particular embodiment, the capsules or solid dosage forms of present invention use paroxetine hydrochloride in a form other than the hemihydrate, and are formulated under conditions such there is no detectable conversion to hemihydrate during the manufacturing process.

This overcomes the surprisingly discovered problem that, even under relatively dry conditions, paroxetine hydrochloride anhydrate has a tendency to convert at least partially to the hemihydrate during tabletting. Although not dangerous, this creates difficulties in establishing and maintaining a reference standard for regulatory and quality control purposes.

The paroxetine hydrochloride may, for example, be present in an amorphous form or as a crystalline anhydrate and dissolved in a carrier, or in the presence of excipients, which are essentially hydrophobic, or essentially anhydrous, typically containing less than 2%, more especially less than 1.5%, preferably less than 1% by wt., water.

The amount of paroxetine used in each capsule is preferably adjusted such that in a single unit dose there is a therapeutically effective amount of paroxetine. Preferably the unit dose contains from 5 to 100 mg paroxetine (as measured in terms of the free base). More preferably the amount of paroxetine in a unit dose is 10 mg, 20 mg, 30 mg, 40 mg or 50 mg. The most preferred amount of paroxetine in a unit dose is 20 mg.

To achieve the desired unit dose in a capsule where the paroxetine is in solution in the carrier, the paroxetine needs to be soluble in the carrier to an extent that allows a sufficient concentration so that the selected capsule volume can contain the desired unit dose. In addition to being able to dissolve paroxetine, the solvent must be compatible with the capsule material and physiologically acceptable for administration to a patient.

Since solid paroxetine forms are in general only sparingly soluble in common solvents, the solvents which are acceptable for use in capsules and for administration to patients need to be subjected to routine solubility testing to confirm that they can maintain an appropriate concentration of paroxetine. In addition, higher loadings of a paroxetine form in a suitable solvent may be achieved by using conventional physical techniques such as heating, shaking and sonication. Alternatively good solvents for paroxetine may be used in small amounts as cosolvents to solubilize paroxetine in liquids that are acceptable for capsule use but in which paroxetine is poorly soluble. Solubilising agents such as the polysorbates, the poloxamers, cyclodextrins, ionic and non-ionic surface active agents, for example Pluronic F60 and Sorbitan esters may also be used to enhance the solubility of paroxetine hydrochloride in solvents acceptable for capsule use but in which paroxetine is poorly soluble.

The term “oral swallow capsule” most suitably denotes a capsule having a maximum volume of 0.86 ml. Preferred capsules according to the present invention have a maximum volume of about 0.45 ml and more especially may lie in the range 0.2 to 0.4 ml, although capsules as small as 0.14 ml are also provided by the invention. A typical capsule at the upper end of the size range acceptable for pharmaceutical use (Soft Gel Size 14 Oblong) has a volume of 0.86 ml. For a 10 mg dose of paroxetine (as free base) 11.11 mg of paroxetine hydrochloride is needed, which in a volume of 0.86 ml requires a concentration of 12.9 mg/ml or 1.29 % w/v. Therefore it is preferred that the solvent which is used has a solubility of at least 10 mg/ml for paroxetine hydrochloride and more preferably the solubility should be at least 25 mg/ml. However larger capsule sizes such as Hard Shell Size 00 (0.95 ml capacity), Supro A (0.68 ml) and Softgel Size 12 Oblong (1.01 ml) may be used when appropriate to provide higher drug dose with the same formulation.

This level of solubility rules out many solvents conventionally used as liquid carriers for encapsulated drugs, such as the plant oils Sunflower, Safflower, Peanut, Soybean, Cottonseed, Corn, Castor, Apricot seed, Olive, Wheat germ, Sesame, Evening Primrose and Canola (Rapeseed) oil, and also Mineral oil and liquid paraffin. Other well known liquid carriers such as Miglyol (810 and 812), Oleic acid, Ethyl Oleate, Span 80 and 85, Labrafac lipophile, Plurol Oleique and Peceol (Glyceryl oleate) also show less than 10 mg/ml solubility.

The present inventors have now identified certain solvents and solvent systems which exhibit the required levels of solubility. Solvents that show a useful solubility include Propylene Carbonate, Triacetin, Glycerol, Lauroglycol, Propylene glycol, PEG 300, Glycofurol, PEG 400, IPA, Span 20, Transcutol, Labrasol, Labrafil, Olepal, Glyceryl Linoleate (Maisine 35-1) and Pharmasolve. For physiological suitability it may be desirable to use such solvents with a cosolvent such as ethanol. The present invention makes use of these solvents and solvent systems as well as of functional equivalents thereof which can be identified using the techniques taught herein.

The present inventors have found that an especially effective means to solubilise paroxetine, particularly the hydrochloride, especially as the hemihydrate, in a liquid, semi-solid or solid carrier, in particular oils and lipids, is to use a solubilising agent, such as N-methyl-2-pyrrolidone (Pharmasolve, International Speciality Products, Texas, USA) as a cosolvent.

Accordingly in a preferred embodiment of this invention, paroxetine, optionally as the free base but more typically as a pharmaceutically acceptable salt such as the hydrochloride is dissolved in a solubilising agent and then blended with an oil or lipid carrier before filling capsules.

The invention also provides as a novel formulation a solution of paroxetine, optionally as the free base but more typically as a pharmaceutically acceptable salt such as the hydrochloride in a blend of a solubilising agent and a lipid and/or oil.

By use of a solubilising agent it is possible to solubilise paroxetine in oils and lipids previously regarded as unsuitable solvents, such as soybean oil, sunflower oil, and arachis oil.

Also by the same means paroxetine may dissolved in lipids, especially lipids derived from natural materials, such coconut oil-derived glycerides, Cithrol 4DL (PEG-8 dilaurate). Examples of coconut oil-derived glycerides include Labrasol and Labrafac CM10 (Gattefosse, France) which are C₈/C₁₀ polyglycolised glycerides from coconut oil having a hydrophilic:lipophilic balance of 14 and 10 respectively.

Formulations based on a solubilising agent and oils/lipids are preferably formulated with at least one antioxidant to maintain stability of the solution on storage. If it desired to use the solutions for filling capsules then the compatibility of the solution with the capsule material must be investigated.

The present invention in a further aspect makes use of supersaturated solutions, for example in solid or semi-solid solvents such as fats and waxes. These may readily be prepared by heating and exhibit high stability because of inter alia their very high viscosity.

Preferably, the solvents used in carrying out the invention contain less than 2%, more especially less than 1.5%, preferably less than 1%, water, or are essentially hydrophobic.

The solution may optionally contain one or more antioxidants such as the tocopherols, ascorbic acid, ascorbic palmitate, thiodipropionic acid, bis hydroxy toluene (BHT), bis hydroxy anisole (BHA), gallic acid, propyl/octyl/dodecyl gallate, benzyl alcohol and nordihydroguaiaretic acid with or without the addition of pH modifiers and chelating agents such as citric acid and EDTA.

The capsule shell may be of any conventional material that is stable to the liquid carrier and solute, for example hard and soft gelatin capsules and starch capsules. In addition to resisting the solvent action of the liquid carrier attention must be paid to the pH of the liquid within the capsule. For example soft gels have a pH limit of 2.5-7.5. Since the addition of paroxetine hydrochloride to a solvent system tends to lower the pH by at least 1 unit, then in general solvent systems with a pH of below 3.5 are not preferred.

According to a further aspect of the invention, the capsules have an enteric resistant coating or incorporate enteric resistant materials in the capsule shell, such that the paroxetine is not discharged in the acidic conditions of the stomach. The object of this is to prevent any undesired uncontrolled precipitation of the paroxetine from solution, and to enable its absorption characteristics to be modified if desired by presenting it to the intestinal mucosa in non-aqueous solution.

The liquid carrier may be present in the capsule as a flowable liquid, as a viscous liquid or semi-solid or as a gel. The viscosity characteristics may be varied by initial choice of solvent or by appropriate use of cosolvents or thickening agents.

A liquid carrier, or a solid or semi-solid carrier that has been softened or made flowable by heating, with dissolved paroxetine may be filled into capsules using conventional capsulation technology.

It may be desirable to use paroxetine hydrochloride in a form other than the hemihydrate, which is formulated into capsules or solid dosage forms under conditions such there is no detectable conversion to hemihydrate during the manufacturing process. The paroxetine hydrochloride may, for example, be present in an amorphous form or as a crystalline anhydrate.

This may be achieved for example by the use of either excipients or carriers which are essentially anhydrous (that is to say, they contain less than 2%, more especially less than 1.5%, preferably less than 1% water) or which are essentially hydrophobic. The capsules and solid dosage forms are then preferably packaged with a desiccant in order to prevent conversion of anhydrate to hemihydrate on storage.

Accordingly, the present invention also provides a process for the preparation of paroxetine hydrochloride anhydrate capsules or solid dosage forms free of detectable hemihydrate which is characterised by the use of conditions such there is no detectable conversion of the anhydrate to hemihydrate during the manufacturing process. Such conditions can be achieved by the use of essentially anhydrous/hydrophobic excipients and/or carriers under conditions of low relative humidity

Examples of excipients with the necessary low moisture content include materials such as dibasic calcium phosphate anhydrous, anhydrous lactose, monosaccharide sugars eg mannitol, disaccharide sugars eg lactitol, powdered cellulose, pregelatinised starch and similar materials. Dibasic calcium phosphate anhydrous is commercially available in a pharmaceutically acceptable grade, eg A-TAB (Rhone Poulenc).

Examples of liquid and semi-solid excipients with the necessary hydrophobicity include materials such as polyglycolised glycerides eg Gelucire 44/14; complex fatty materials of plant origin eg theobroma oil, carnauba wax; plant oils eg peanut, olive, palm kernels, cotton, corn, soya; hydrogenated plant oils eg peanut, palm kernels, cotton, soya, castor, coconut; natural fatty materials of animal origin eg beeswax, lanolin, fatty alcohols eg cetyl, stearyl, lauric, myristic, palmitic, stearic; esters eg glycerol stearate, glycol stearate, ethyl oleate, isopropyl myristate; solid interesterified semi-synthetic glycerides eg Suppocire, Witepsol; liquid interesterified semi-synthetic glycerides eg Miglyol 810/812,; amide or fatty acid alcolamides eg stearamide ethanol, diethanolamide of fatty coconut acids; polyoxyethylene glycols eg PEG 600, PEG 4000.

Liquids and semi-solids having suitable solubility characteristics to act as carriers for dissolved paroxetine, and having similar hydrophobicity to the above liquid excipients, include Labraphil, a liquid interesterified semi-synthetic glyceride, and PEG 400, a polyoxyethylene glycol.

The above solid and liquid excipients may be blended with carriers of suitable solubility for paroxetine disclosed above and if necessary cosolvents to obtain solutions of paroxetine with anhydrous/hydrophobic properties. Carriers already having suitable anhydrous/hydrophobic properties may be blended directly with paroxetine, again using cosolvents where necessary to promote dissolution. The formulations may be filled into capsules, such as gelatin capsule shells, or cellulose capsule shells of intrinsically low moisture content (eg Shionogi Qualicaps, <3%).

Liquid interesterified semi-synthetic glycerides commercially available in a pharmaceutically acceptable grade include Labrafil M 2125CS (Gattfosse). In a particular process of the invention, paroxetine hydrochloride anhydrate is mixed with Labrafil M 2125CS (Gatfosse) to produce a formulation for encapsulation in a hard or soft gelatin capsule.

Paroxetine in the form of the hydrochloride anhydrate may be prepared according to the procedures outlined in WO 96/24595. Suitable procedures for preparing paroxetine include those mentioned in U.S. Pat. Nos. 4,009,196, 4,902,801, 4,861,893 and 5,039,803 and PCT/GB 93/00721.

The present invention also provides solid dosage forms of paroxetine for oral swallow use in which paroxetine is dissolved in a polymeric carrier. These forms include tablets, pellets, spheroids, granules, lozenges and gels containing paroxetine in solid solution.

To achieve the desired unit dose in for example a melt extruded tablet where the paroxetine is in solution in the polymer carrier, the paroxetine needs to be soluble in the polymer carrier or a solvent/cosolvent that is soluble in the polymer carrier to an extent that allows a sufficient concentration so that the selected tablet size and volume can contain the desired unit dose. In addition to being able to dissolve paroxetine, the solvent/cosolvent must be compatible with the polymer carrier material and physiologically acceptable for administration to a patient.

When the solid dosage form is granules or pellets then a plurality of granules or pellets may be collected in an aggregation that as a whole constitutes a unit dose. The granules or pellets may be used as a fill for capsules or pressed, optionally with binders or excipients, into tablet form.

Since solid paroxetine forms are in general only sparingly soluble in common solvents, the solvents/co-solvents and carriers which are acceptable for use in the above dosage forms and for administration to patients need to be subjected to routine solubility testing to confirm that they can maintain an appropriate concentration of paroxetine. In addition, higher loadings of a paroxetine form in a suitable solvent may be achieved by using conventional physical techniques such as heating, shaking and sonication. Alternatively good solvents for paroxetine may be used in small amounts as cosolvents to solubilize paroxetine in polymers that are acceptable for melt extrusion, melt granulation, gel formulation use but in which paroxetine is poorly soluble. Solubilising agents such as the polysorbates, the poloxamers, cyclodextrins, ionic and non-ionic surface active agents, for example Pluronic F60 and Sorbitan esters may also be used to enhance the solubility of paroxetine hydrochloride in solvents acceptable for polymers used to produce solid solution systems in forms of tablet, pellet, granule, spheroid use but in which paroxetine is poorly soluble.

It is preferred that the polymer and/or solvent which are used have a solubility of at least 10 mg/ml for paroxetine hydrochloride and more preferably the solubility should be at least 25 mg/ml.

In general the use of polymers in this invention to produce semi-solid/solid solution system offer a broad flexibility of use. Beside filling into hard/soft gelatin capsules they may be used to make melt extruded system such as tablets, pellets, spheroid and any other shape depending on the shape of the extruder die, can be injection moulded into different shapes and/or melt granulated to produce pellets or granules. Alternatively the granules can be milled and pressed into tablets and other shapes depending on the shape and design of the press die.

Examples of the pharmaceutical polymers used for the above applications are film forming and thermoplastic polymers that are generally approved substances listed in international Pharmacopoeias such as polyethylene oxide water soluble resins, ethoxylated glycerides and triglycerides, cetyl esters, cetyl palmitate, glyceryl esters, polyvinyl acetate, cellulose, lanolin based product, vinyl resins, latex product, carbowax polyethylene glycols, gelatin (protein), ethylene oxide/glycol such as ethylene glycol, glycol ethers and ethanolamines, unipol polymers, polypropylene resins, silicone products, saturated polyglycolysed glycerides, glyceryl behenate, glyceryl palmitostearate, semisynthetic glycerides and vinyl acetate monomers. The function(s) of these polymers will be as a drug carrier and/or solubiliser and/or binder and/or permeability enhancers.

Solvents that show a useful solubility for paroxetine, such as Propylene Carbonate, Triacetin, Glycerol, Lauroglycol, Propylene glycol, PEG 300, Glycofurol, PEG 400, IPA, Span 20, Transcutol, Labrasol, Labrafil, Olepal, Glyceryl Linoleate (Maisine 35-1) and Pharmasolve mentioned previously, may be used as cosolvents to assist solubilisation of paroxetine in the solide, semi-solid and polymeric carriers mentioned above. For physiological suitability it may be desirable to use such solvents with another cosolvent such as ethanol. The present invention makes use of these solvents and solvent systems as well as of functional equivalents thereof which can be identified using the techniques taught herein.

An appropriate lanolin derivative e.g. ethoxy-75 lanolin is commercially available in a pharmaceutical grade e.g. Solan E (Croda). In a particular process of the invention, paroxetine hydrochloride hemihydrate is dissolved in Pharmasolve and mixed with molten Solan E in a suitable blender to form granules on cooling, drying, sifting then solid solution tablet upon compression.

A polyglycolised glyceride is commercially available in a pharmaceutically acceptable grade, e.g. Gelucire 44/14 (Gattfosse). In a particular process of the paroxetine invention, paroxetine hydrochloride hemihydrate is dissolved in Pharmasolve and then mixed with molten Gelucire 44/14 to form a melt extrudate in forms of a tablet and/or pellet on cooling.

Polyethylene glycols of different molecular weights are commercially available in a pharmaceutically acceptable grade e.g. PEG 4000 (Union Carbide Corp & BASF). In a particular process of the invention, Paroxetine hydrochloride hemihydrate is dissolved in PEG 300 and then mixed with molten PEG 4000 to form melt extruded materials which on cooling as solid solution may be converted into forms of tablets and/or pellets.

The solid solution may optionally contain one or more antioxidants such as the tocopherols, ascorbic acid, ascorbyl palmitate, thiodipropionic acid, bis hydroxy toluene (BHT), bis hydroxy anisole (BHA), gallic acid, propyl/octyl/dodecyl gallate, benzyl alcohol and nordihydroguaiaretic acid with or without the addition of pH modifiers and chelating agents such as citric acid and EDTA.

According to a further aspect of the invention, the solid dosage form may have an enteric resistant coating such that paroxetine is not discharged in the acidic conditions of the stomach. The object of this is to prevent any undesired uncontrolled precipitation of the paroxetine from solution, and to enable its absorption characteristics to be modified if desired by presenting it to the intestinal mucosa in an aqueous solution.

The solid solution/semi-solid systems presented in this invention can be coated with suitable polymer that can be used with melt granulation or hot melt coating such as Precirol ATO 5 (Glyceryl palmito stearate) for taste-masking paroxetine and/or enterically coated with methacrylic acid copolymer C (e.g. Eudragit L 30 D-55).

The semi-solid or gel formulation can also be optionally capsulated. The viscosity characteristics of the semi-solid or gel may be varied by initial choice and amount of solvent or by appropriate use of cosolvents or thickening agents.

The semi-solid or gel carrier with dissolved paroxetine may be filled into capsules using conventional capsulation technology.

Self-supporting solid of paroxetine solution can be successfully prepared in forms of tablet, pellets, spheroid, granules using Solan E, Gelucire, higher molecular weights of PEG's and gel based on gelatin with different cosolvents constituents.

For example, the paroxetine is first dissolved in co-solvent constituents, such as PEG 300, Pharmasolve and water/ethanol (using sufficient mixing to assure complete wetting/solubilisation). The resultant mixture is then preheated and added to a suitable portions of a melted polymer such as Gelucire 44/14 (melting point 42-46 C), Solan E (melting point 45-50 C), PEG 6000 (melting point 55-63 C), PEG 4000 (melting point 50-58 C) or Gelatin (gelatin in liquid co-solvents melted between 50-55 C). The samples may then be left at ambient condition for resolidification of the polymer to occur. A shaping device may then be used to produce solid dosage forms as tablets, pellets, spheroids and gels. The drug molecule dissolved in the polymer during the melting phase will remain dissolved in the finished product as a solid solution. With gelatin based formulations, transparent solid solutions containing dissolved drug are produced.

As mentioned above, itt may be desirable to use paroxetine hydrochloride in a form other than the hemihydrate, which is formulated into self-supporting solid dosage forms under conditions such there is no detectable conversion to hemihydrate during the manufacturing process. The paroxetine hydrochloride may, for example, be present in an amorphous form or as a crystalline anhydrate.

As already described, this may be achieved for example by the use of either excipients or polymeric carriers which are essentially anhydrous (that is to say, they contain less than 2%, more especially less than 1.5%, preferably less than 1% water) or which are essentially hydrophobic.

Therapeutic uses of the paroxetine formulations of this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression trichotillomania, dysthymia, and substance abuse, referred to below as “the disorders”.

Accordingly, the present invention also provides:

the use of paroxetine dissolved in a carrier to manufacture oral swallow capsules or solid dosage forms for the treatment or prophylaxis of one or more of the disorders;

a method of treating the disorders which comprises administering an effective or prophylactic amount of paroxetine as a solution in a carrier in an oral swallow capsule or solid dosage form to a person suffering from one or more of the disorders;

a method of treating the disorders which comprises administering an effective or prophylactic amount of paroxetine as a solution in a liquid formulation of the invention to a person suffering from one or more of the disorders.

The formulations of this invention may also be used where appropriate for veterinary treatment of animals.

The invention is illustrated by the following Examples:

(Paroxetine Anhydrous Free Base 10.0 mg is Equivalent to 11.38 mg Paroxetine HCl—Conversion Factor From Paroxetine HCl to Paroxetine Anhydrous Base is 0.8787).

In Examples 1-10, paroxetine is dissolved in a carrier, optionally assisted by a cosolvent, and filled into capsules.

| | Excipient | mg per capsule |
|---|---|---|
| Example 1. | | |
| | Paroxetine hydrochloride | 22.22 |
| | Polyethylene Glycol 400 | 450.0 |
| Capsule | Size 11 Oblong Soft Gel | |
| Example 2. | | |
| | Paroxetine hydrochloride | 22.22 |
| | Polyethylene Glycol 400 | 400.0 |
| | Ethanol | 45.0 |
| Capsule | Size 0 Hard Shell, banded | |
| Example 3. | | |
| | Paroxetine hydrochloride | 22.22 |
| | Propylene Glycol | 350.0 |
| Capsule: | Size 8 Oblong Soft gel | |
| Enteric Coat | Methacrylic Acid | 32.0 |
| | Copolymer Type C | |
| | Propylene Glycol | 8.0 |
| Example 4. | | |
| | Paroxetine hydrochloride | 22.22 |
| | Fractionated coconut oil | 300.0 |
| | Polyethylene Glycol 400 | 150.0 |
| | Polysorbate 80 | 50.0 |
| Capsule: | Size 11 Oblong Soft gel | |
| Example 5. | | |
| | Paroxetine hydrochloride | 22.22 |
| | Glycerol | 100.0 |
| | Propylene Glycol | 100.0 |
| | Propyl gallate | 0.3 |
| Capsule: | Size 5 Oblong Soft gel | |
| Example 6. | | |
| | Paroxetine hydrochloride | 22.22 |
| | Glycofurol | 100.0 |
| | Polyethylene glycol 300 | 50.0 |
| | Citric acid | 1.5 |
| | BHT | 0.02 |
| Capsule: | Size 4 Oblong Soft gel | |
| Example 7. | | |
| | Paroxetine hydrochloride | 22.22 |
| | Pharmasolve | 50.0 |
| | High Purity Cotton Seed Oil | 150.0 |
| | Propyl gallate | 0.2 |
| Capsule: | Size 4 Oblong Soft gel | |
| Example 8. | | |
| | Paroxetine hydrochloride | 22.22 |
| | Polyethylene Glycol 400 | 50.0 |
| | Pharmasolve | 10.0 |
| | Citric Acid | 2.0 |
| Capsule | Size 3 Oval Soft Gel | |
| Example 9. | | |
| | Paroxetine hydrochloride | 22.22 |
| | Lauroglycol 400 | 100.0 |
| | Pharmasolve | 10.0 |
| | Citric Acid | 2.0 |
| Capsule | Size 3 Hard Shell, banded | |
| Example 10. | | |
| | Paroxetine hydrochloride | 22.22 |
| | Polyethylene Glycol 400 | 50.0 |
| | Pharmasolve | 10.0 |
| | Citric Acid | 2.0 |
| Capsule | Starch Capil | |

In Example 11, paroxetine is dissolved in a hydrophobic carrier.

| Example 11 | mg |
|---|---|
| Paroxetine hydrochloride † | 22.22 |
| Labrafil M 2125CS | 227.78 |
| Capsule weight | 250.00 |

In Examples 12-30, paroxetine was dissolved in a cosolvent, and then blended with a molten polymer. Clear paroxetine solutions were obtained before solidification of the polymers.

| | | | |
|---|---|---|---|
| Example 12 | Paroxetine HCL | 22.76 | mg |
| | PEG 300 | 200.00 | mg |
| | PEG 4000 | 300.00 | mg |
| | dl alfa tocopherol | 0.1% | w/w |
| Tablet | Ascorbyl Palmitate | 0.1% | w/w |
| Example 13 | Paroxetine Hydrochloride | 45.52 | mg |
| | Gelucire 44/14 | 227.78 | mg |
| Tablet | Pharmasolve | 100.00 | mg |
| Example 14 | Paroxetine Hydrochloride | 22.76 | mg |
| Tablet | Gelucire 44/14 | 227.78 | mg |
| Example 15 | Paroxetine Hydrochloride | 6828 | mg |
| | Solan E | 350.00 | mg |
| | (ethoxy 75 lanolin) | | |
| Tablet | Pharmasolve | 150.00 | mg |
| Example 16 | Paroxetine Hydrochloride | 22.76 | mg |
| Tablet | PEG 1450 | 227.78 | mg |
| Example 17 | Paroxetine Hydrochloride | 22.76 | mg |
| Tablet | PEG 4000 | 227.78 | mg |
| Example 18 | Paroxetine HCL | 19.91 | mg |
| | PEG 300 | 200.00 | mg |
| | PEG 1450 | 300.00 | mg |
| | dl alfa tocopherol | 0.1% | w/w |
| Tablet | Ascorbyl Palmitate | 0.1% | w/w |
| Example 19 | Paroxetine Hydrochloride | 22.76 | mg |
| Tablet | Suppocire DM | 227.78 | mg |
| Example 20 | Paroxetine HCL | 73.96 | mg |
| | Gelatine | 100.00 | mg |
| | Purified water | 350.00 | mg |
| | Pharmasolve | 150.00 | mg |
| | Polysorbate 80 | 1 | drop |
| Gel | Methyl Paraben | 0.2% | w/w |
| Example 21 | Paroxetine HCL | 42.67 | mg |
| | Gelatine | 50.00 | mg |
| | Purified water | 200.00 | mg |
| | Propylene Glycol | 400.00 | mg |
| | Propyl Gallate | 0.1% | w/w |
| | Ascorbic Acid | 0.1% | w/w |
| Gel | Polysorbate 80 | 1 | drop |
| Example 22 | Paroxetine HCL | 113.79 | mg |
| | Gelatine | 50.00 | mg |
| | Purified water | 200.00 | mg |
| | Pharmasolve | 200.00 | mg |
| | Propylene Glycol | 200.00 | mg |
| | Polysorbate 80 | 1 | drop |
| Gel | Methyl Paraben | 0.2% | w/w |
| Example 23 | Paroxetine HCL | 102.41 | mg |
| | Gelatine | 50.00 | mg |
| | Purified water | 200.00 | mg |
| | Pharmasolve | 200.00 | mg |
| | Ethanol | 200.00 | mg |
| | Polysorbate 80 | 1 | drop |
| Gel | Methyl Paraben | 0.2% | w/w |
| Example 24 | Paroxetine HCL | 28.45 | mg |
| | Gelatine | 50.00 | mg |
| | Purified water | 200.00 | mg |
| | Ethanol | 200.00 | mg |
| | Propylene Glycol | 200.00 | mg |
| Gel | Polysorbate 80 | 1 | drop |
| Example 25 | Paroxetine HCL | 45.52 | mg |
| | Gelatine | 50.00 | mg |
| | Purified water | 200.00 | mg |
| | Propylene Glycol | 400.00 | mg |
| | PEG 300 | 50.00 | mg |
| Gel | Polysorbate 80 | 1 | drop |
| Example 26 | Paroxetine HCL | 11.38 | mg |
| | Gelatine | 50.00 | mg |
| | Purified water | 500.00 | mg |
| | Propylene Glycol | 100.00 | mg |
| Gel | Polysorbate 80 | 1 | drop |
| Example 27 | Paroxetine HCL | 28.45 | mg |
| | Gelatine | 50.00 | mg |
| | Purified water | 300.00 | mg |
| | Propylene Glycol | 300.00 | mg |
| Gel | Polysorbate 80 | 1 | drop |
| Example 28 | Paroxetine HCL | 68.28 | mg |
| | Gelatine | 50.00 | mg |
| | Purified water | 300.00 | mg |
| | Pharmasolve | 150.00 | mg |
| | Propylene Glycol | 150.00 | mg |
| Gel | Polysorbate 80 | 1 | drop |
| Example 29 | Paroxetine HCL | 79.65 | mg |
| | Gelatine | 50.00 | mg |
| | Purified water | 300.00 | mg |
| | Pharmasolve | 150.00 | mg |
| | Ethanol | 150.00 | mg |
| | Polysorbate 80 | 1 | drop |
| Gel | Methyl Paraben | 0.2% | w/w |
| Example 30 | Paroxetine HCL | 17.07 | mg |
| | Gelatine | 50.00 | mg |
| | Purified water | 300.00 | mg |
| | Propylene Glycol | 150.00 | mg |
| | Ethanol | 150.00 | mg |
| Gel | Polysorbate 80 | 1 | drop |

In Examples 31-44, paroxetine is initially dissolved in Pharmasolve and the resultant solution is blended with oil and lipid carriers, so that the paroxetine is dissolved in the carrier to give liquid formulations that may be capsulated (36-42) and also provided with an enteric coating (43-45)

| | | | Appearance | Stability |
|---|---|---|---|---|
| | | | of System/ | of Pxt |
| | Composition | | Solution\hat{ } | solution* |
| Example 31 | Labrasol | 2.25 mL | clear pale | clear very |
| | Pharmasolve | 0.25 mL | yellow | pale pink |
| | Drug | 125 mg | solution | solution |
| Example 32 | Cithrol 4DL | 2.25 mL | clear pale | clear pale |
| | Pharmasolve | 0.25 mL | yellow | pink |
| | Drug | 125 mg | solution | solution |
| Example 34 | Sunflower oil | 2.25 mL | clear pale | no change |
| | Pharmasolve | 0.25 mL | yellow | clear v. pale |
| | Drug | 125 mg | solution | yellow |
| | | | | solution |
| Example 35 | Soybean oil | 2.25 mL | clear pale | no change |
| | Pharmasolve | 0.25 mL | yellow | clear pale |
| | Drug | 125 mg | solution | yellow |
| | | | | solution |
| Example 36 | Arachis oil | 2.25 mL | clear pale | no change |
| | Pharmasolve | 0.25 mL | yellow | clear v. pale |
| | Drug | 125 mg | solution | yellow |
| | | | | solution |

*stored at RT for 25 days (visual observation)
\hat{ }at the time of preparation (fresh samples)

1. An oral swallow capsule containing paroxetine dissolved in a carrier.

2. An oral swallow capsule comprising a capsule shell containing paroxetine as the free base or a pharmaceutically acceptable salt or solvate thereof in solution in a liquid or solid carrier.

3. A capsule according to claim 2 in which the carrier is a liquid solvent present in the capsule as a flowable liquid, as a viscous liquid or semi-solid or as a gel.

4. A capsule according to claim 2 in which the carrier is a solid or semi-solid solvent.

5. A capsule according to claim 4 in which the solid or semi-solid solvent is selected from natural and synthetic fats and waxes, and film-forming and thermoplastic polymers.

6. An oral swallow solid dosage form containing paroxetine dissolved in a solid, semi-solid or gel carrier.

7. A solid dosage form comprising tablets, pellets, spheroids, granules, lozenges or gels in which paroxetine is present as a solid solution in a polymeric carrier.

8. Capsules and solid dosage forms according to any one of claims 1 to 7 which are coated to assist in administration of the active ingredient.

9. Capsules and solid dosage forms according to claim 8 which are coated with coatings to delay or control release of paroxetine and/or coatings for taste-masking.

10. Capsules and solid dosage forms according to any one of claims 1 to 9 in which paroxetine is used as the hydrochloride hemihydrate or anhydrate.

11. Capsules and solid dosage forms according to any one of claims 1 to 10 in which paroxetine is used as paroxetine hydrochloride in a form other than the hemihydrate, which is formulated under conditions such there is no detectable conversion to hemihydrate during the manufacturing process.

12. Capsules and solid dosage forms according to claim 11 in which the paroxetine hydrochloride is used in an amorphous form or as a crystalline anhydrate.

13. Capsules and solid dosage forms according to claim 12 in which the paroxetine hydrochloride is dissolved in a carrier which is essentially hydrophobic or anhydrous.

14. Capsules and solid dosage forms according to claim 12 or 13 in which the paroxetine hydrochloride is dissolved in a carrier in the presence of excipients, which are essentially hydrophobic or anhydrous.

15. A pharmaceutical formulation comprising a solution of paroxetine in a blend of a solubilising agent and a lipid and/or oil.

16. A process for preparing a formulation according to claim 15 which comprises dissolving paroxetine in a solubilising agent and blending the resultant solution with a lipid and/or oil.

17. The use of paroxetine dissolved in a carrier to manufacture oral swallow capsules or solid dosage forms according to any one of claims 1 to 14 for the treatment or prophylaxis of one or more of the disorders;

18. A method of treating the disorders which comprises administering an effective or prophylactic amount of paroxetine as a solution in a carrier in an oral swallow capsule or solid dosage form according to any one of claims 1 to 14 to a mammal suffering from one or more of the disorders.

19. A method of treating the disorders which comprises administering an effective or prophylactic amount of paroxetine as a solution in a formulation according to claim 15 to a mammal suffering from one or more of the disorders.
